(19) United States
(12) Patent Application Publication
(10) Pub. No.: US 2007/0055377 A1
(43) Pub. Date: Mar. 8, 2007

(54) Bone implants and methods

(75) Inventors: David A. Hanson, St. Louis Park, MN (US); Ross A. Longhini, West Lakeland, MN (US); Daniel D. McPhillips, Ham Lake, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/534,249

(22) Filed: Sep. 22, 2006

Related U.S. Application Data
(63) Continuation of application No. 10/468,197, filed on Aug. 15, 2003, now abandoned.
(60) Provisional application No. 60/269,777, filed on Feb. 16, 2001.

(51) Int. Cl. A61F 2/44

(52) U.S. Cl. 623/17.11

(57) ABSTRACT

The disclosure provides implants and methods for bone fusion procedures. In some embodiments, the implants are particularly advantageous for use between opposing vertebral bodies to facilitate stabilization or arthrodesis of an intervertebral joint. The implants includes, at least, a support component that provides structural support during fusion. In a typical embodiment, the implants also include a growth component. A growth component provides an environment conductive to new bone growth between the bones being fused. Several unique configuration to enhance fusion, instruments for insertion and methods for insertion use are also disclosed.

This application is being filed as a PCT international patent application in the names of David A Hanson, Ross A. Longhini, and Daniel D. McPhillips, all U.S. residents and citizens, on 19 Feb. 2002, designating all countries.

### FIELD OF THE INVENTION

This invention pertains to bone implants and procedures. Specifically, the invention provides bone implants and methods to facilitate fusion of bone. The invention is particularly suited for stabilization or fusion of the intervertebral disc space between adjacent vertebrae.

### BACKGROUND OF THE INVENTION

Chronic back problems cause pain and disability for a large segment of the population. Frequently, the cause of back pain is traceable to diseased disc material between opposing vertebrae. When the disc material is diseased, the opposing vertebrae may be inadequately supported, resulting in persistent pain. Surgical techniques have been developed to remove the diseased disc material and fuse the joint between opposing vertebral bodies. Stabilization and/or arthrodesis of the intervertebral joint can reduce the pain associated with movement of a diseased intervertebral joint. Spinal fusion may be indicated to provide stabilization of the spinal column for a wide variety of spine disorders including, for example, structural deformity, traumatic instability, degenerative instability, post-resection iatrogenic instability, etc.

Generally, fusion techniques involve removal of the diseased disc and packing the void area with a suitable matrix for facilitating a bony union between the opposing vertebral bodies.

Surgical devices for facilitating interbody fusion are known. Some devices are positioned external to the intervertebral joint during the fusion process. Other devices are positioned within the intervertebral joint. Devices positioned within the joint space typically distract the joint space and provide stabilization by causing tension on the annular ligament and other supporting tissues surrounding the joint space. Examples of devices positioned within the joint space are disclosed in, for example, U.S. Pat. Nos. 5,458,638, 5,489,307, 5,055,104, 5,026,373, 5,015,247, 4,961,740, 4,743,256 and 4,501,269, the entire disclosures of which are incorporated herein by reference. Some systems use both external fixation and internal fixation devices.

Regardless of the type or location of the fusion device, a bone graft is often used to facilitate new bone growth. The surface area, configuration, orientation, surface, texture and deformity characteristics of an implant or bone graft placed in the disc space can affect the stability of the joint during fusion and thus affect the overall success of a fusion procedure.

Accordingly, the present invention is directed to unique implants or bone grafts that can be inserted at a fusion site, with or without other stabilizing systems.

### SUMMARY OF THE INVENTION

At various locations throughout the specification, lists of examples are provided. It should be noted that the examples are provided for illustrative purposes and are not intended to limit the scope of the invention.

The invention provides implants, instruments and methods for fusion of bones.

### BRIEF DESCRIPTION OF THE DRAWINGS

FIG. 1a is an exploded perspective view of one embodiment of an implant according to the invention;

FIG. 1b is a perspective view of the implant of FIG. 1a;

FIG. 1c is a plan view of a first bearing surface of the implant of FIG. 1a;

FIG. 1d is a plan view of a first end of the implant of FIG. 1a;

FIG. 1e is a plan view of a first side of the implant of FIG. 1a;

FIG. 2a is an exploded perspective view of an alternative embodiment of an implant according to the invention;

FIG. 2b is a perspective view of the implant of FIG. 2a;

FIG. 2c is a plan view of a first side of the implant of FIG. 2a;

FIG. 2d is a plan view of a first end of the implant of FIG. 2a;

FIG. 2e is a plan view of a first bearing surface of the implant of FIG. 2a;

FIG. 3a is a perspective of an alternative embodiment of an implant according to the invention;

FIG. 3b is a plan view of a side of the implant of FIG. 3a;

FIG. 3c is a plan view of a first end of the implant of FIG. 3a;

FIG. 3d is a plan view of a first bearing surface of the implant of FIG. 3a;

FIG. 4a is a perspective view of an alternative embodiment of an implant according to the invention;

FIG. 4b is a plan view of a side of the implant of FIG. 4a;

FIG. 4c is a plan view of a side of the implant of FIG. 4a rotated 90° from the view in FIG. 4b;

FIG. 4d is a plan view of a bearing surface of the implant of FIG. 4a;

FIG. 5a is a perspective view of an alternative embodiment of an implant according to the invention;

FIG. 5b is a plan view of a side of the implant of FIG. 5a;

FIG. 5c is a plan view of a side of the implant of 5a rotated 90° from the view of FIG. 5b;

FIG. 5d is a plan view of a first bearing surface of the implant of FIG. 5a;

FIG. 6a is a perspective view of an alternative embodiment of an implant according to the invention;

FIG. 6b is a plan view of a side of the implant of FIG. 6a;

FIG. 6c is a plan view of a side of the implant of FIG. 6a rotated 90° from the view of FIG. 6b;

FIG. 6d is a plan view of a first bearing surface of the implant of FIG. 6a;

FIG. 7a is a perspective view of an alternative embodiment of an implant according to the invention;

FIG. 7b is a plan view of a side of the implant of FIG. 7a;

FIG. 7c is a side view of the implant of FIG. 7a rotated 90° from the view of FIG. 7b;

FIG. 7d is a plan view of a first bearing surface of the implant of FIG. 7a.

FIG. 8a is a perspective view of an alternative embodiment of an implant according to the invention;

FIG. 8b is a plan view of a first side of the implant of FIG. 8a;

FIG. 8c is a side view of the implant of FIG. 8a rotated 90° from the view of FIG. 8b;

FIG. 8d is a plan view of a first bearing surface of the implant of FIG. 8a;

FIG. 9a is a perspective view of an alternative embodiment of an implant according to the invention;

FIG. 9b is a plan view of a first side of an implant of FIG. 9a;

FIG. 9c is a plan view of a first end of the implant of FIG. 9a;

FIG. 9d is a plan view of a first bearing surface of the implant of FIG. 9a;

FIG. 10a is a perspective view of an alternative embodiment of an implant according to the invention;

FIG. 10b is a plan view of a first side of the implant of FIG. 10a;

FIG. 10c is a plan view of a first bearing surface of the implant of FIG. 10a;

FIG. 10d is a plan view of a first end of the implant of FIG. 10a;

FIG. 11a is a perspective view of an alternative embodiment of an implant according to the invention;

FIG. 11b is a plan view of a first side of the implant of FIG. 11a;

FIG. 11c is a side view of the implant of FIG. 11a rotated 90° from the view of FIG. 11b;

FIG. 11d is a plan view of a first bearing surface of the implant of FIG. 11a;

FIG. 11e is a profile view of one embodiment of a pin of an anchoring arrangement according to the invention;

FIG. 11f is an alternative embodiment of a pin of an anchoring arrangement according to the invention;

FIG. 12a is a perspective view of an alternative embodiment of an implant according to the invention;

FIG. 12b is a plan view of a first bearing surface of the implant of FIG. 12a;

FIG. 12c is a longitudinal cross-section view of the implant of FIG. 12a taken through line 12c-12c of FIG. 12b;

FIG. 12d is a plan view of a first end of the implant of FIG. 12a;

FIG. 12e is a profile view of one embodiment of an instrument suitable for use with the implant of FIG. 12a;

FIG. 12f is a cross-section view through line 12f-12f of FIG. 12e;

FIG. 13a is a perspective view of an alternative embodiment of an implant according to the invention;

FIG. 13b is a plan view of a first side of the implant of FIG. 13a;

FIG. 13c is a plan view of a first bearing surface of the implant of FIG. 13a;

FIG. 13d is a plan view of a second side of the implant of FIG. 13a;

FIG. 14a is a perspective of an alternative embodiment of an implant according to the invention;

FIG. 14b is a plan view of a first bearing surface of the implant of FIG. 14a;

FIG. 14c is a plan view of a side of the implant of FIG. 14a;

FIG. 14d is a plan view of a side of the implant of FIG. 14a rotated 90° from the view in FIG. 14c;

FIG. 14e is a perspective view of the implant of FIG. 14a without the presence of a growth component;

FIG. 14f is a plan view of the implant of FIG. 14a as shown in FIG. 14d, without the presence of a growth component;

FIG. 15a is a perspective view of one embodiment of a cap according to the invention;

FIG. 15b is a plan view of one side of the cap of FIG. 15a;

FIG. 15c is a plan view of a first bearing surface of the cap of FIG. 15a;

FIG. 15d is a plan view of the outer wall of the cap of FIG. 15a;

FIG. 16a is a profile view of one embodiment of an implant insertion tool according to the invention;

FIG. 16b is a profile view of the implant insertion tool of FIG. 16a, rotated 90° around axis A_{T};

FIG. 17a is a profile view of an alternative embodiment of an implant insertion tool;

FIG. 17b is a profile view of the implant insertion tool of FIG. 17a rotated 90° along axis A_{T};

FIG. 18a is a perspective view of an alternative embodiment of an implant according to the invention;

FIG. 18b is a plan view of a first bearing surface of the implant of FIG. 18a; and

FIG. 18c is a plan view of a side of the implant of FIG. 18a.

### DETAILED DESCRIPTION OF THE INVENTION

The present invention is directed to fusion of bones. The invention provides natural and synthetic bone implants that can function as a bone graft between adjacent bones to be fused. The implants of the invention include several unique arrangements, configurations and components to facilitate fusion and maintain stability during the fusion process.

The implants and methods of the invention can be used in a variety of bone fusion procedures. In some embodiments, the invention may be particularly advantageous for intervertebral stabilization or arthrodesis of the intervertebral disc space between adjacent vertebrae. Accordingly, for purposes of description herein, the invention will be described by reference to intervertebral fusion procedures. However, this description is for exemplary purposes only and should not be construed to limit the intended scope of use of the disclosed implants. In the case of vertebral fusion, the implants and methods of the invention can be used to fuse cervical, thoracic, lumbar or lumbo-sacral vertebrae.

In general, the implants and methods of the invention are directed to facilitating greater continuity between the bone formed at the fusion site and the bones fused. The implants are also designed to provide greater structural support at the fusion site to maintain stability and alignment at the fusion site, to reduce healing time and optimize the structural integrity of the new bone formed at the fusion site. The implants of the invention can also facilitate the ease of implanting and positioning implants at a fusion site.

The implants can be prepared from natural materials, synthetic materials, or a combination of natural and synthetic materials. As used herein, “natural material” means “bone” and includes bone harvested from humans or animals. Additionally, the implants can be prepared from products made from bone, such as chips, putties, and other similar bone products. “Bone” may further include heterologous, homologous and autologous (i.e., xenograft, allograft, autograft) bone derived from, for example, fibula, tibia, radius, ulna, humerus, cranium, calcaneus, tarsus, carpus, vertebra, patella, ilium, etc. In some embodiments, human source bone is preferred for human applications.

The bone of an implant can be cancellous and/or cortical. In one embodiment, cortical bone is present in the implant to provide, support, stabilization or alignment at the fusion site, while cancellous bone can be present to provide a matrix to support new bone growth. That is, the cortical portions of an implant can provide strength for support and the cancellous portion provide increased surface area to facilitate tissue-growth, vascularization and deposition of new bone.

Cortical implant material can be obtained from known long bones, such as the humerus, radius, ulna, tibia, femur, fibula, etc. Cancellous material can be obtained from the patella, distal-condyles, tibial plateau, femoral head, etc. Cranial, pelvic (e.g. iliaccrest) and patellar bone can advantageously provide both cortical and cancellous bone in a single piece. Indeed, these sources can provide an implant having cancellous bone surrounded on opposing sides by cortical bone. In one embodiment, the inventors have discovered that cranial bone provides a superior implant for fusion of adjacent vertebral bodies.

In some embodiments, the “support” portion (component) of an implant of the invention is provided by cortical bone or a natural or synthetic material having biomechanical and biological characteristics similar to cortical bone. The support portion provides support, stabilization, and facilitates alignment at the fusion site, etc. The “growth” portion (component) of the implant can be provided by a bone growth matrix, cancellous bone, etc. The growth portion provides a matrix to support new bone growth. One preferred bone growth component that can also provide some support is cancellous bone. “Porous” synthetic materials can also act as a supporting, growth component. As used herein, a “porous synthetic material” includes, for example, porous titanium, porous ceramics, porous stainless steel, etc. Such porous materials permit the growth portion and the support portion of the implant to overlap.

In some embodiments, the growth component of the implant can be prepared from cancellous bone or alternatively a “bone growth matrix” shaped into any one of the advantageous configurations of growth components disclosed herein. As used herein a “bone growth matrix” is a material that facilitates new bone growth. Suitable bone support matrices can be resorbable or nonresorbable and osteoconductive or osteoinductive. Examples of suitable matrices according to the invention include synthetic materials, such as Healos™, available from Orquest, Mountain View, Calif.; or any of a variety of bone morphogenic proteins (BMPs).

“Synthetic materials” include non-bone materials such as titanium, stainless steel, porous titanium, ceramic, carbon fiber, silicon, methylmethacrylate, polytetrafluoroethylene, polycarbonate urethane (PEEK) and other materials suitable for use as an orthopedic implant. Further, the materials may include any of the above synthetic materials combined with a natural bone material. For example, the material may comprise a combination of bioglass and bone chips or bone chips with a bonding agent. As stated above, an implant of the invention can consist solely of a synthetic material. In other applications, a synthetic material may be used in combination with cancellous bone.

An implant of the invention can have one of several configurations including a single component or a plurality of components. In one embodiment, the implants have first and second bearing surfaces, which in use are positioned adjacent opposing vertebrae. The bearing surfaces can include an engaging surface having a surface texture that enhances stability at the bone-implant interface and reduces the likelihood of motion during the fusion process. Examples of engaging surfaces suitable for the invention include ridges, knurls, grooves, teeth, serrations, etc.

The implants can be sized for a particular application. For example, for stabilizing a lumbar disc space, the implant preferably has a height dimension “H” of about 2 mm to 30 mm, a width dimension “W” of about 6 mm to 40 nm and a length dimension “L” of about 10 mm to 40 mm. Other sizes will be appreciated as being within the scope of the invention after review of the present disclosure.

In some embodiments, the implants can be stabilized at the fusion site through the use of an anchoring arrangement, comprising an anchor such as pins, screws, etc., that can pass through bores formed within the implant to anchor the implant to the bones to be fused. Anchoring arrangements can be used with or without engaging surfaces. Coupling arrangements can also be used to couple multiple components of the implant together. A coupling arrangement can include a coupler such as pins, screws, slots, ridges, etc., and a bore formed in the implant components for receiving the coupler. In some embodiments, the anchor and the coupler can be prepared from bone, such as cortical bone. Suitable anchoring arrangements and coupling arrangements, as well as methods for manufacturing, are disclosed in, for example, U.S. Pat. Nos. 5,968,047 and 5,868,749, the entire disclosures of which are incorporated herein by reference. Couplers and anchors can also be prepared from synthetic materials using known methods.

Natural or synthetic bone implants of the invention can be manufactured using procedures known in the art. Methods for preparing natural bone implants are disclosed in for example, U.S. Pat. Nos. 6,033,438; 5,968,047; 5,585,116; 5,112,354; and 5,439,684; the entire disclosures of which are incorporated herein by reference.

Instruments and methods for preparing a bone fusion site for receiving an implant and for positioning an implant at the site are known in the art and include, for example, U.S. Pat. Nos. 5,989,289; 5,968,047; and co-pending U.S. Ser. Nos. 09/631,502 and 09/611,237. The entire disclosure of each of these patents and patent applications are incorporated herein by reference.

### DETAILED DESCRIPTION OF THE ILLUSTRATED EMBODIMENTS

The implants, instruments and methods of the invention will now be described by reference to the several drawing figures. The functional features of the implants of the invention can be embodied in any of a number of specific configurations. It will be appreciated, however, that the illustrated embodiments are provided for descriptive purposes and should not be used to limit the invention. In addition, in many embodiments, cortical and cancellous bone are used. It will be appreciated from an understanding of the present invention that the cortical or support portions of the implants can be substituted with synthetic materials.

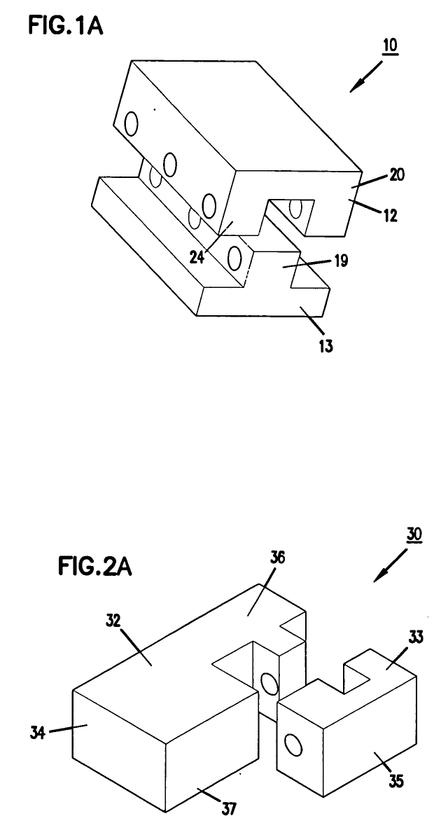

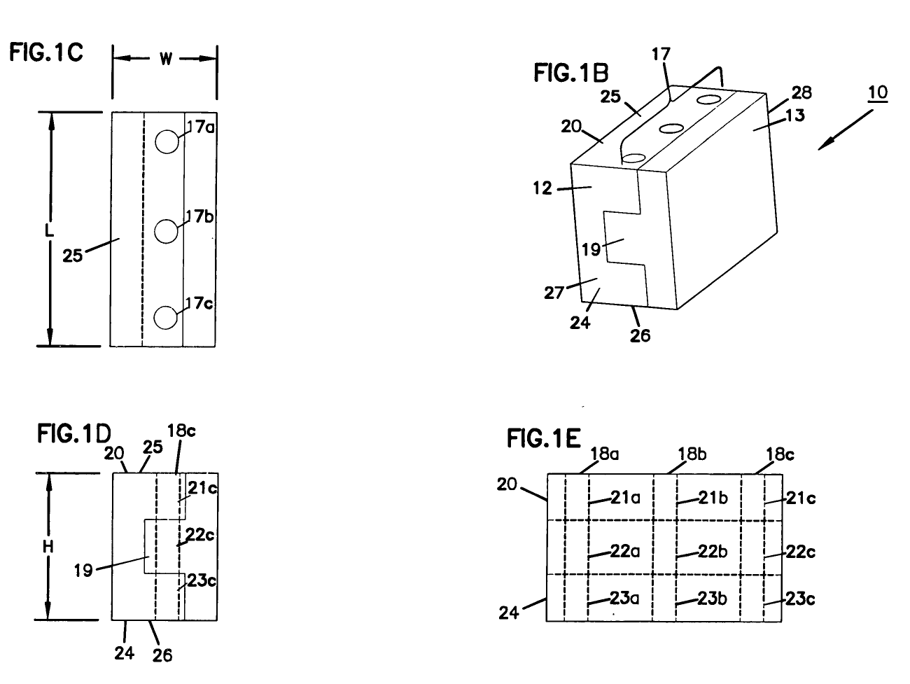

FIGS. 1a-1e illustrate one embodiment of an implant according to the invention having an overall rectilinear configuration. As illustrated, implant 10 comprises a body including a first component 12 and a second component 13. First component 12 can be a support component and second component 13 can be a growth component. Thus, for example, the first component 12 can be derived from support material such as cortical bone; and the second component 13 can be derived from growth material such as cancellous bone. Synthetic materials or composite materials may be used as discussed previously.

First component 12 and second component 13 can be coupled together with a coupling arrangement 17. In the illustrated embodiment, coupling arrangement 17 comprises pins 17a-17c inserted through channels 18a-18c which comprise bores 21a-21c from first side 20 of first component 12, bores 22a-22c passing through overlapping portion 19 of second component 13 and bores 23a-23c passing through second side 24 of first component 12.

Implant 10 includes a first bearing surface 25, a second bearing surface 26, a first end 27 and a second end 28. In this embodiment, implant 10 has a height dimension H a width dimension W and a length dimension L. Thus, as an example, when used as an intervertebral implant between adjacent vertebrae, bearing surface 25 can be positioned adjacent the bottom endplate of a first vertebrae and bearing surface 26 adjacent the top endplate of a second vertebrae.

Thus, support component 12 provides stability to maintain the adjacent vertebrae in a spaced apart relationship the distance of height H and growth component 13 provides a matrix for new bone growth.

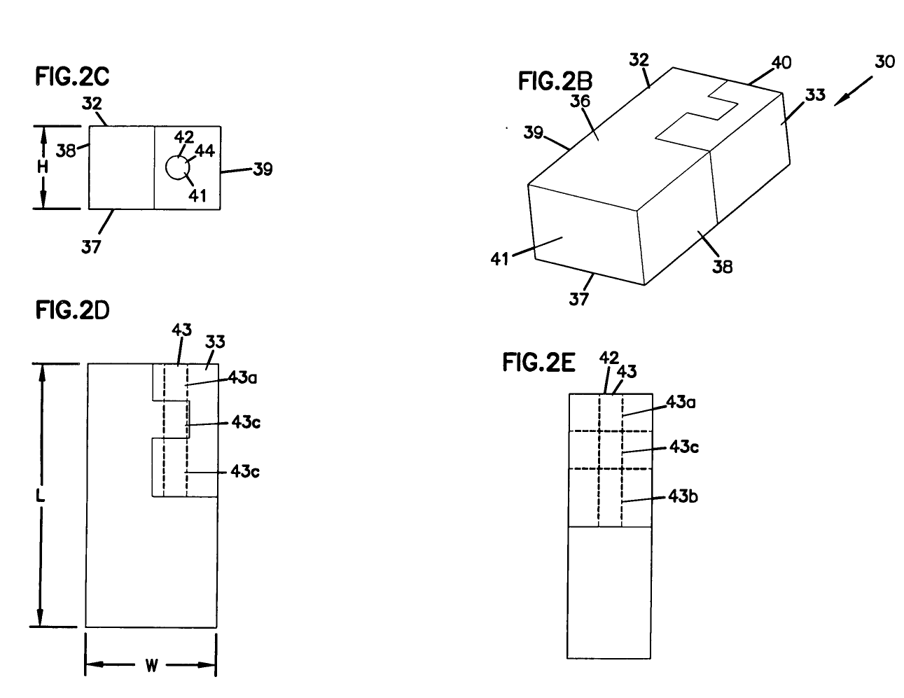

Referring now to FIGS. 2a-2e, in an alternative embodiment, implant 30 includes a body having a first component 32 and a second component 33. The first component 32 can be a support component and the second component can be a growth component. Implant 30 also includes a first bearing surface 36, a second bearing surface 37, a first side surface 38, a second surface 39, a first end surface 40 and a second end surface 41.

FIG. 2c is a side plan view illustrating that first component 32 and second component 33 can be fixed together with a coupling arrangement 42 comprising a channel 43 and pin 44. Channel 43 comprises bores 43a and 43b in first component 32 and bore 43c in second component 33.

Implant 30 has a height dimension H, width dimension W, and length dimension L. In the illustrated embodiment, for use as an intervertebral implant, implant 30 can be positioned such that first bearing surface 36 is adjacent the inferior endplate of a first vertebrae and second bearing surface 37 adjacent the inferior endplate of a second vertebrae.

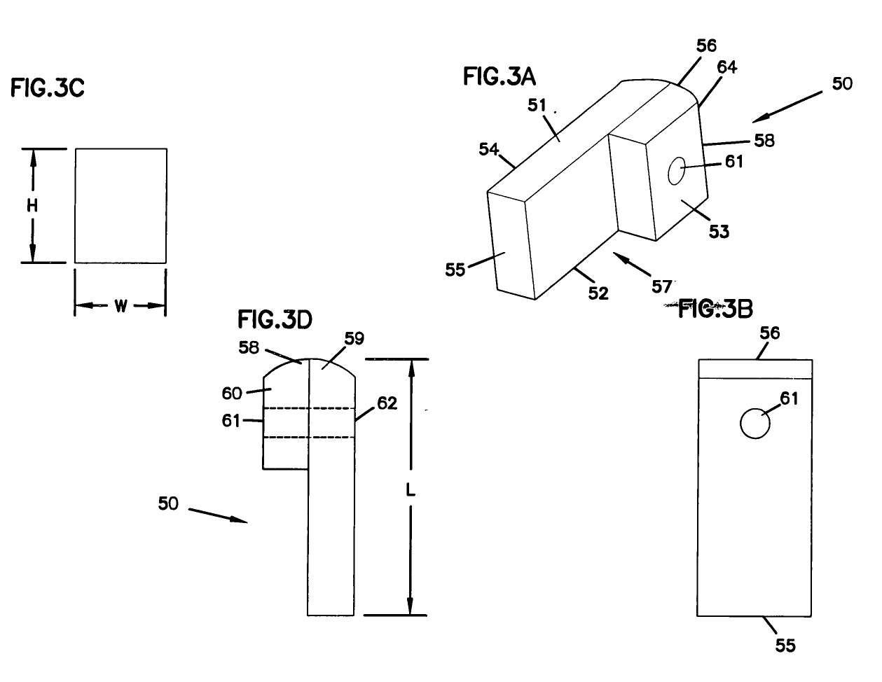

Referring now to FIGS. 3a-3d, in an alternative embodiment, implant 50 comprises a body 58 having a first bearing surface 51, a second bearing surface 52, a first side surface 53, a second side surface 54, a first end surface 55 and a second end surface 56. An L-shaped void 57 in implant 50 can be filled with a growth component such as cancellous bone or a bone growth matrix as described above. As shown in FIG. 3d, body 58 can comprise a first component 59 and a second component 60 maintained in a fixed relationship by coupling arrangement 61 including a pin 62 passed through a channel. Alternatively, body 58 could be a single piece. It will be appreciated that in the illustrated embodiment, the second end of implant 56 has a rounded surface 64. This provides for the implant to follow the contours of a bone, for example the anterior face of a lumbar vertebrae. In addition to other advantages, this embodiment allows for reduced surgical dissection for placement of the implant.

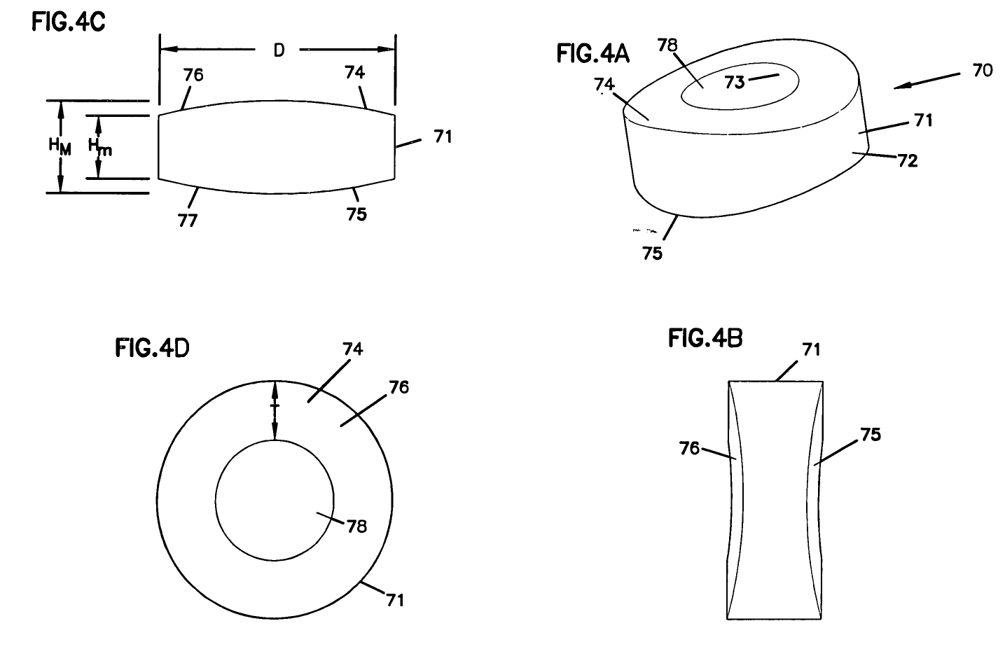

Referring now to FIGS. 4a-4d, there is illustrated an alternative embodiment of an implant 70 according to the invention. As will be appreciated, implant 70 comprises a body 71 that is ring shaped having an outer wall 72, inner wall 73, first bearing surface 74 and second bearing surface 75. As seen best in FIG. 4c, first bearing surface 74 is convex 76 across diameter D and second bearing surface 75 is also convex 77 across diameter D. Thus, this embodiment provides bi-convex bearing surfaces 76 and 77.

Dimension T from inner wall 73 to outer wall 72 is preferably about 2 mm to 10 mm, more preferably about 4 mm to 8 mm. The bi-convex bearing surfaces provide implant 70 with a minor height Hₘ and a major height H_{M}. It will be appreciated that while the illustrated embodiment is circular, oval, elliptical rectangular, bi-oval and other shapes can also be used. In this embodiment, body 71 of implant 70 is a supporting component. Thus, implant body 70 can be prepared from cortical bone or a suitable synthetic material. A growth component can be added in the form of a cylindrical shaped piece of cancellous bone to fit within void 78 or void 78 can be filled with some type of bone growth enhancing matrix. The convex surface of implant 70 can advantageously provide for cooperative fit with concave surfaces of the vertebral endplates.

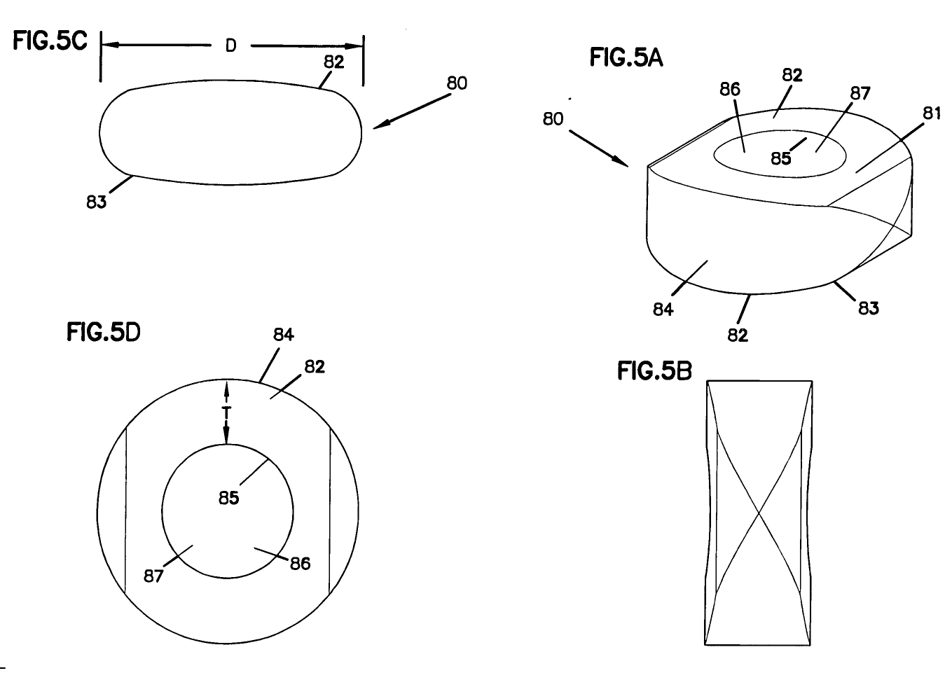

Referring now to FIGS. 5a-5d, in an alternative embodiment, implant 80 comprises a body 81 having a first bearing surface 82, a second bearing surface 83, an outer wall 84, and an inner wall 85. As illustrated best in side view 5c, implant 80 includes multi-radii surfaces. Implant 80 has a diameter D and thickness T between outer wall 84 and inner wall 85. Void 87 can be filled with a growth component or growth enhancing matrix as described above. In addition to the circular configuration shown, implant 80 could also be oval, elliptical, rectangular, etc. The multi-radii surfaces of implant 80 can advantageously provide for enhanced cooperative fit at the lateral margins of the vertebral endplates.

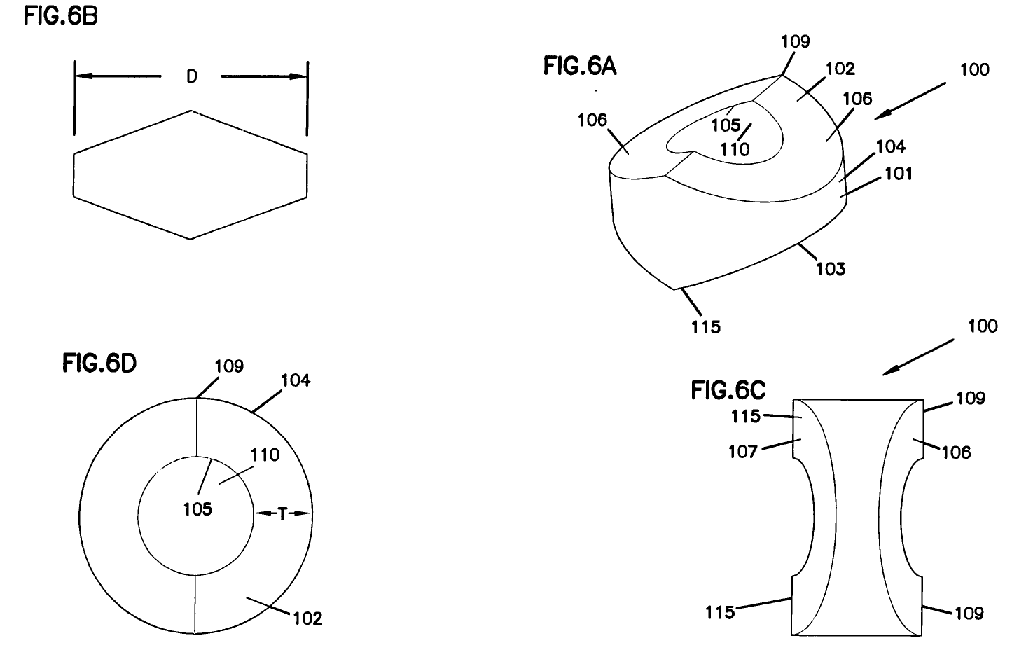

FIGS. 6a-6d illustrate an alternative embodiment of an implant 100. According to this embodiment, implant 100 includes a body 101 having a first bearing surface 102, a second bearing surface 103, an outer wall 104 and an inner wall 105. In the illustrated embodiment, implant 100 is circular, but other configurations as described above could be used. In this embodiment, the first bearing surface 102 includes a first hemi-circular portion 106 and a second hemi-circular portion 107 in a different plane, the planes meeting at apex edge 109. A similar arrangement is present for second bearing surface 103, forming apex 115.

As described for other implants, implant 100 has a diameter D and a wall thickness T extending between outer wall 104 and inner wall 105. Body 101 is a support component that can be prepared from cortical bone or synthetic material. Void 110 can be filled with a growth component comprising cancellous bone or a bone growth matrix.

According to this embodiment, a groove can be formed in each of the opposing vertebral endplates. These grooves are formed at a location to provide for interdigitation with apices 109 and 115.

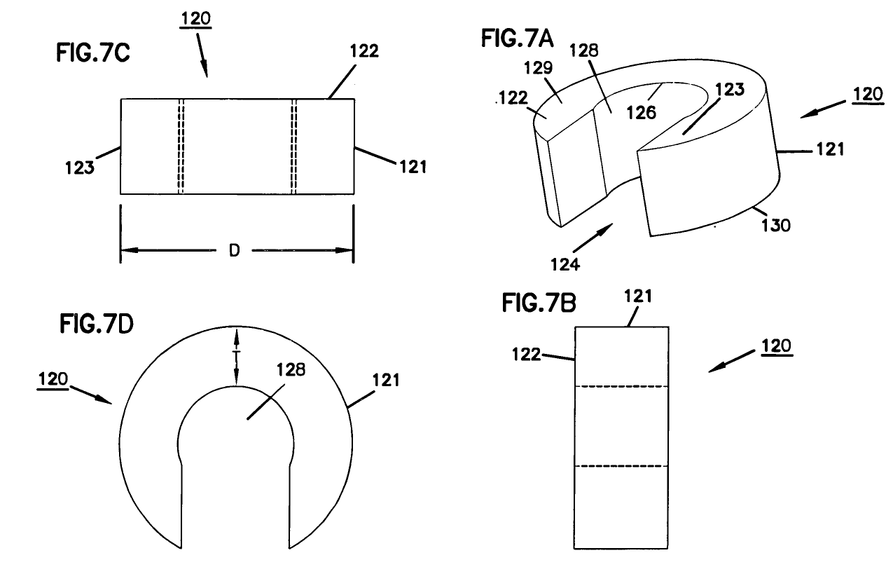

FIGS. 7a-7d illustrate another embodiment of an implant 120 according to the invention. In this embodiment, implant 120 includes a body 121 having a “C-shaped” configuration with a first arm 122 continuous with a second arm 123 having an opening 124 therebetween. In the illustrated embodiment, inner wall 126 forms a center void 128 continuous with opening 124. Implant 120 has a diameter D and a thickness T between outer wall 125 and inner wall 126. Void 128 and opening 124 can be filled with a growth component as previously described.

Implant 120 is particularly advantageous for use in an anterior lumbar interbody fusion or posterior lumbar interbody fusion procedure. During positioning in an intervertebral space, opening 124 can be positioned to face a surgeon. The bearing surfaces 129, 130 are planar. Alternatively, one or both of bearing surfaces 129, 130 could be configured as described for implants 70, 80 and 100.

The configuration of implant 120 provides for easier positioning at a fusion site. In addition, the increased void area 128, 124 provides for a greater volume of a growth component.

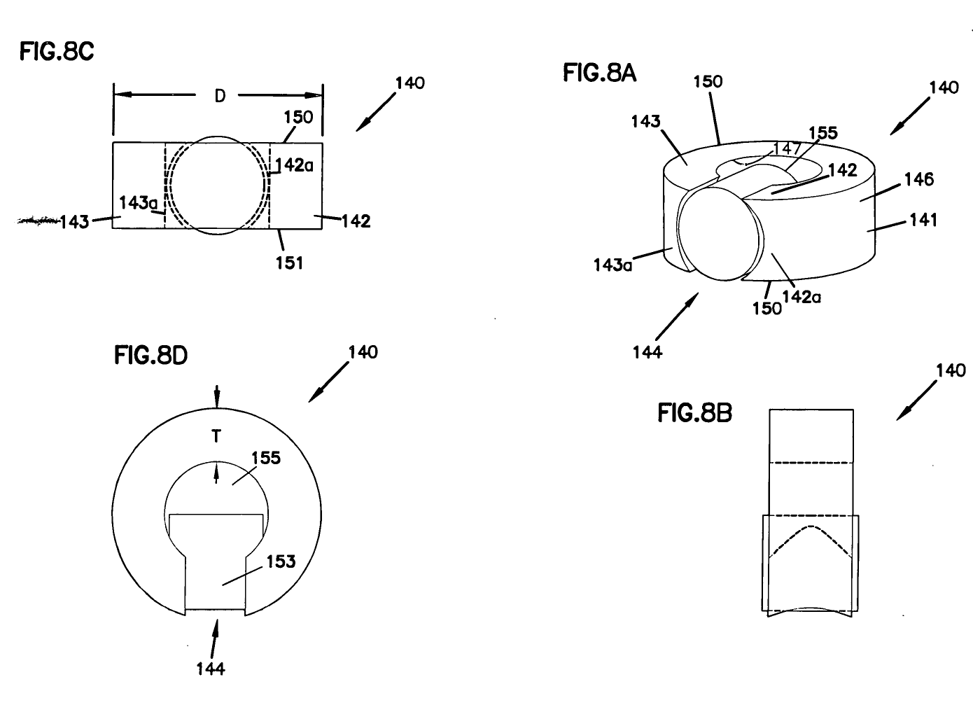

FIGS. 8a-8d illustrate an alternative embodiment of an implant 140. According to this embodiment, implant 140 includes a body 141 having a “C-shaped” configuration comprising a first arm 142 continuous with a second arm 143 forming a space 144 therebetween. Body 141 also includes an external wall 146 and an internal wall 147. As best illustrated in FIGS. 8a and 8c, the facing surfaces of arms 142 and 143 are concave 142a, 143a, respectively. First bearing surface 150 and second bearing surface 151 are planar. However, in an alternative embodiment, one or both of bearing surfaces 150 and 151 could be configured as described for implants 70, 80 or 100.

A central void 155 is bounded by inner wall 147 and is continuous with opening 144 between arms 142 and 143. Thus, body 141 is a support component which can receive a growth component 153 in central void 155. In the illustrated embodiment, growth component 153 can be a dowel of cancellous bone.

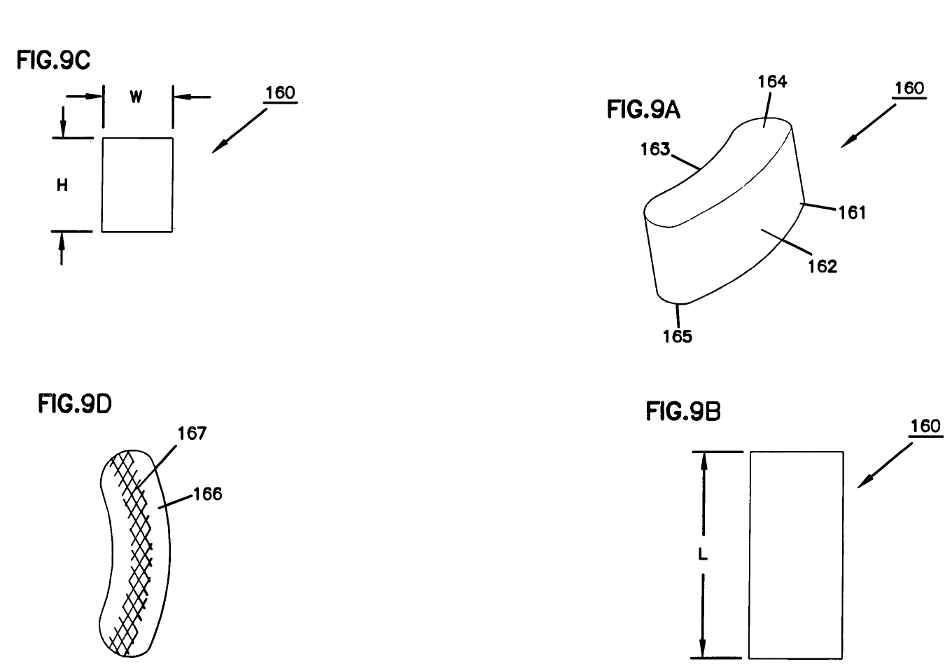

FIGS. 9a-9d illustrate an alternative embodiment of an implant 160 according to the invention. As illustrated, implant 160 comprises a body 161 having a first side surface 162, a second side surface 163, a first bearing surface 164 and a second bearing surface 165. In the illustrated embodiment, implant 160 has a curvilinear configuration. As with other implants of the invention, the height H, width W and length L dimensions can be sized for a particular application. Implant 160 can be advantageously used in a posterior lumbar interbody fusion procedure as well as an anterior lumbar interbody fusion procedure.

In one preferred embodiment, implant 160 can be prepared from cranial bone to provide an outer region of cortical bone 166 and an inner region of cancellous bone 167 as shown diagrammatically in FIG. 9d. It has been determined that the natural continuity of the cortical and cancellous bone of cranial bone has a strength and density that is particularly advantageous for use as an implant for vertebral fusion procedures.

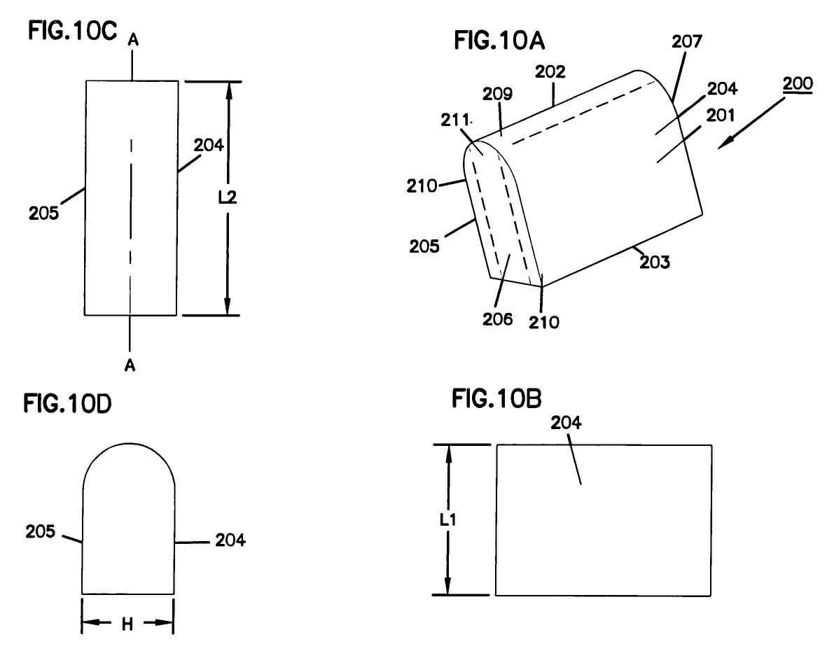

Referring now to FIGS. 10a-10d, implant 200 comprises a body 201 having a first load bearing end or first bearing surface 202, second load bearing end or second bearing surface 203, first side 204, second side 205, first end 206 and second end 207. The first load bearing end or surface 202 is positioned opposite the second load bearing end or surface 203. In this embodiment, length L1 is defined between the first load bearing end or surface 202 and the second load bearing end or surface 203; length L2 is defined between the first end 206 and the second end 207; and height H is defined between the first side 204 and the second side 205.

The implant 200 may be prepared from cortical or cortical cancellous bone such as cranium. In the illustrated embodiment, the first side 204 and the second side 205 comprise support components 210. A growth component 211 is positioned between the first and second support components. Preferably, the growth component is exposed at the first and second load bearing ends or surfaces 202 and 203 to accommodate and promote bone ingrowth. A growth component can also be packed around the implant after placement.

The illustrated embodiment of FIGS. 10a-10d also includes a pivot structure 209 at the first load bearing end or surface 202. The pivot structure may comprise a rounded or convex end as shown. It is contemplated that a pivot structure may also be located at the second load bearing end or surface 203. The pivot structure may be defined by only the growth component positioned between the support components; by only the support components, or by both the growth and support components. The pivot structure assists in rotational placement of the implant 200.

The configuration of implant 200 provides for positioning implant 200 in an intervertebral disc space with first side 204 adjacent an inferior endplate of a first vertebrae and side 205 adjacent a superior endplate of a second vertebrae. Alternatively, sides 204 and 205 can be positioned at an angle from 0° to 90° relative to the horizontal plane of the disc space. Once in position, implant 200 can be rotated around axis A to a position of 90° from the horizontal plane of the disc space such that bearing surface 202 is positioned against the inferior endplate of the first vertebrae and second bearing surface 203 positioned against the superior endplate of the second vertebrae (or vice versa). Implant 200 could also be initially positioned with second side 205 adjacent the inferior endplate and first side 204 adjacent the superior endplate.

This embodiment permits insertion of the implant through a narrower opening with subsequent distraction of the disc space upon rotation.

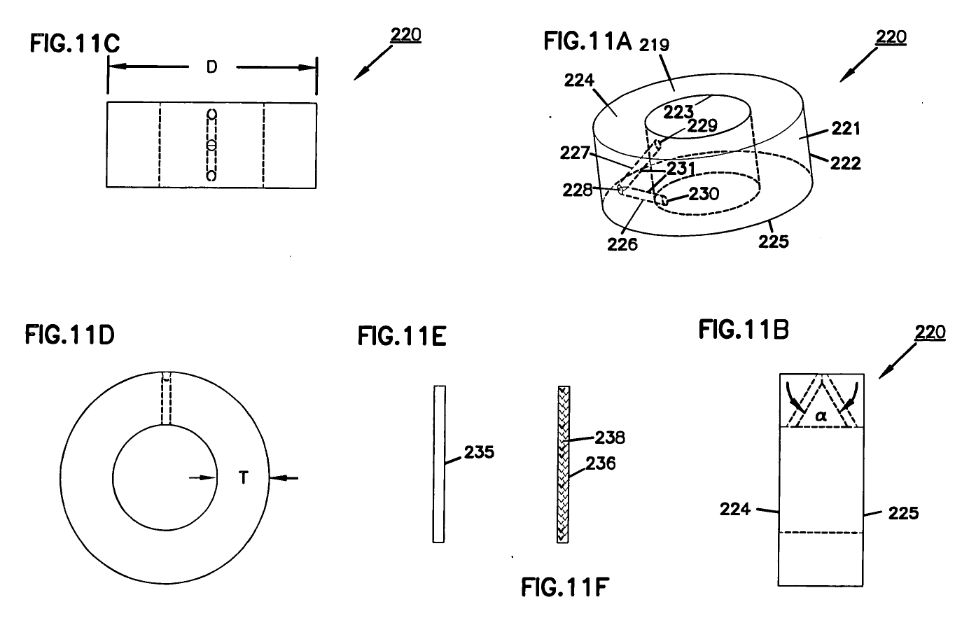

Referring now to FIGS. 11a-11f, implant 220 is illustrated as a circular implant having a body 221 comprising an outer wall 222, inner wall 223, first bearing surface 224, second bearing surface 225 and void 219. Although implant 220 is illustrated as having a circular configuration, it can alternatively have an oval, elliptical rectangular, or other shape.

As shown in phantom lines, in the illustrated embodiment, implant 220 includes a first channel 226 and a second channel 227, each of the channels originating from a single opening 228 at outer wall 222 and passing through to opening 229 at outer wall 223 near first bearing surface 224 and opening 230 near second bearing surface 225 at inner wall 223. (Alternatively, channels 226 and 227 can have distinct openings at outer wall 222.) Channels 226 and 227 are part of an anchoring arrangement 231 to fix implant 220 in position. For example, once positioned in an intervertebral disc space, an anchor such as pins 235 or 236 (FIGS. 11e and 11f) can be passed from opening 228, through channels 226 and 227 and driven into the endplates of the vertebrae to anchor the implant in position. The angle α between channel 227 and 226 can be between 0° and 90°, preferably about 20° to 60°.

Body 221 is a supporting component and can be made from previously described materials. A growth component, such as cancellous bone or a bone growth matrix can be positioned in void 226. As with other implants disclosed herein, bearing surfaces 224 and 225 need not be planar but, for example, one or both can be configured as described for implants 70, 80 and 100.

In one embodiment, pins 235 and 236 can be prepared from cortical bone using procedures known in the art, for example, as described in U.S. Pat. Nos. 5,968,047 and 5,868,749. Alternatively, pins can be prepared from materials such as polylactic acid, poly-lactic-glycolic acid, stainless steel titanium, etc. As illustrated, in one embodiment, pin 236 can include barbs 238 to provide greater resistance against displacement of the pin once positioned in the channel.

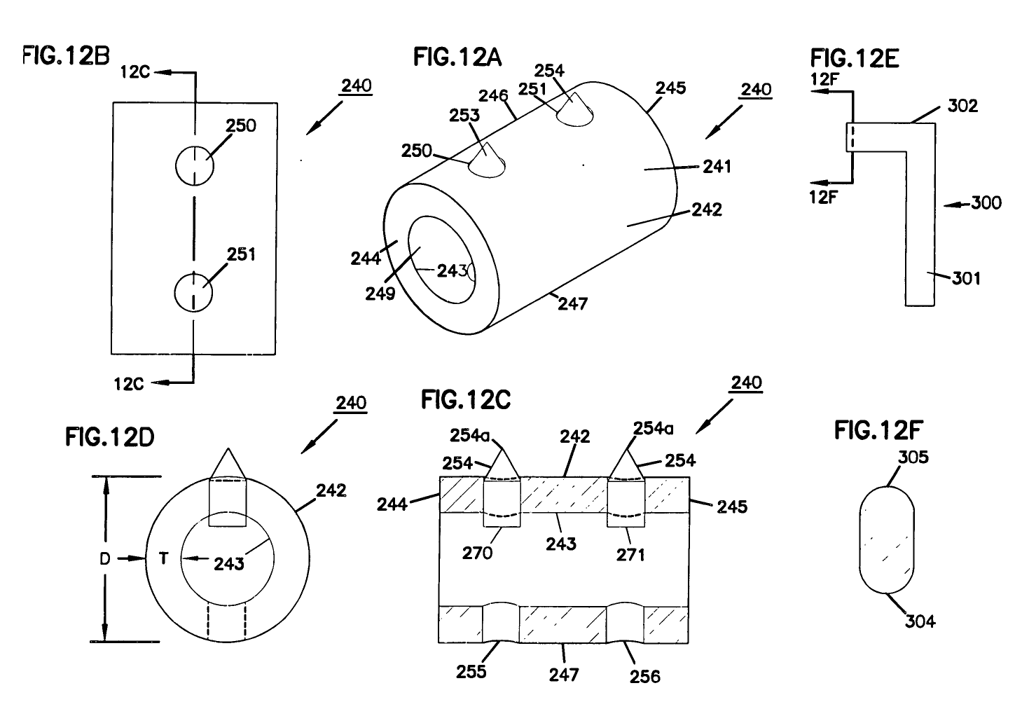

Referring to FIGS. 12a-12c, implant 240 comprises a body 241 having an outer wall 242, inner wall 243, first end 244, second end 245 and bearing surfaces 246 and 247. Inner wall 243 provides a cylindrical chamber 249 within implant 240. At bearing surface 246 there are openings 250 and 251 passing from outer wall 242 to chamber 249. Pins 253 and 254 can each have piercing tips 253a and 254a, respectively. As illustrated in the cross-section view of FIG. 12c, second bearing surface 247 can also have openings 255 and 256 similar to those at bearing surface 246.

Thus, in use, pins 253, 254 or similar pins, can be passed through openings 250, 251, 255 and 256 and retracted into chamber 249. After implant 249 is positioned into an intervertebral disc space, the pins can be extended from the chamber beyond outer wall 242 to engage the endplates of the vertebrae by a tool that wedges against the internal ends of the pins (e.g., 270, 271) or has a cam configuration which when rotated drives the pins from chamber into the endplates of the vertebrae.

For example, FIG. 12e is a side view of an instrument 300 having a handle 301 and working end 302. As seen in the cross-section view of FIG. 12f, when the oval configuration of working end 302 is rotated within chamber 249, the apices 304, 305 will protract the pins from the chamber into the vertebral endplates.

After the pins are engaged into or against the endplates, chamber 249 can be filled with a growth component such as cancellous bone or a bone growth matrix.

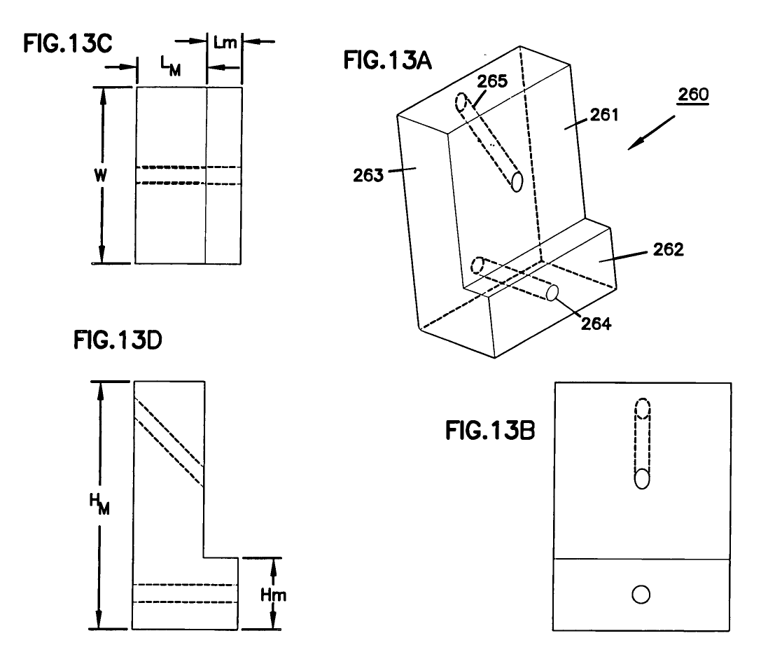

FIGS. 13a-13d illustrate a jig 260 to facilitate positioning and fixation of an implant. According to this embodiment, jig 260 includes a body 261 having a first guide region 262 and a second guide region 263. A first channel 264 passes through first guide region 262 and a second channel 265 passes through second guide region 263.

Jig 260 can be advantageously used, for example, in an anterior interbody fusion procedure. According to this embodiment, first guide region 262 can be inserted into an intervertebral disc space with second guide region 263 extending inferiorly or superiorly adjacent the anterior face of the vertebrae. In this orientation, first guide region 262 can distract the intervertebral disc space distance Hₘ. A drill can then be guided through channel 265 into the anterior face of the vertebra to form a bore in the pre-placed growth component. An anchor (as described previously) can then be passed through the bore to anchor the growth component 260 to the vertebrae.

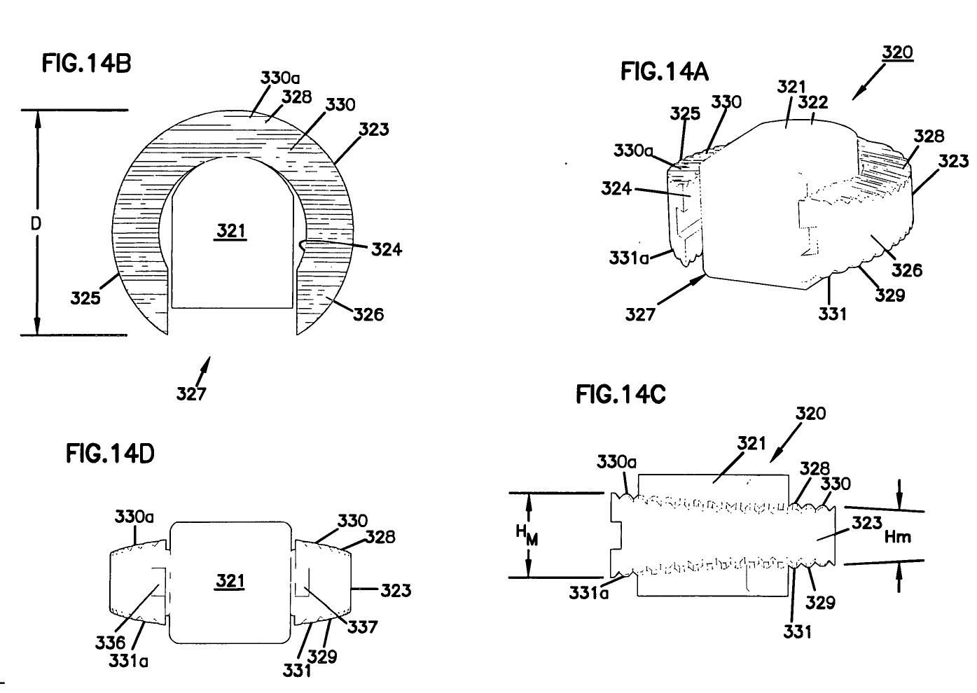

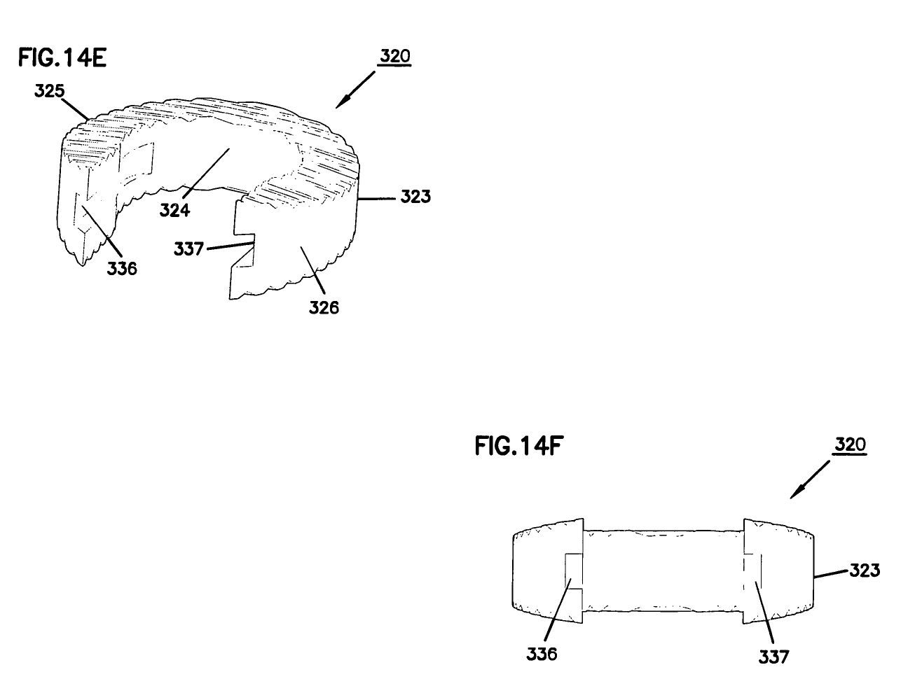

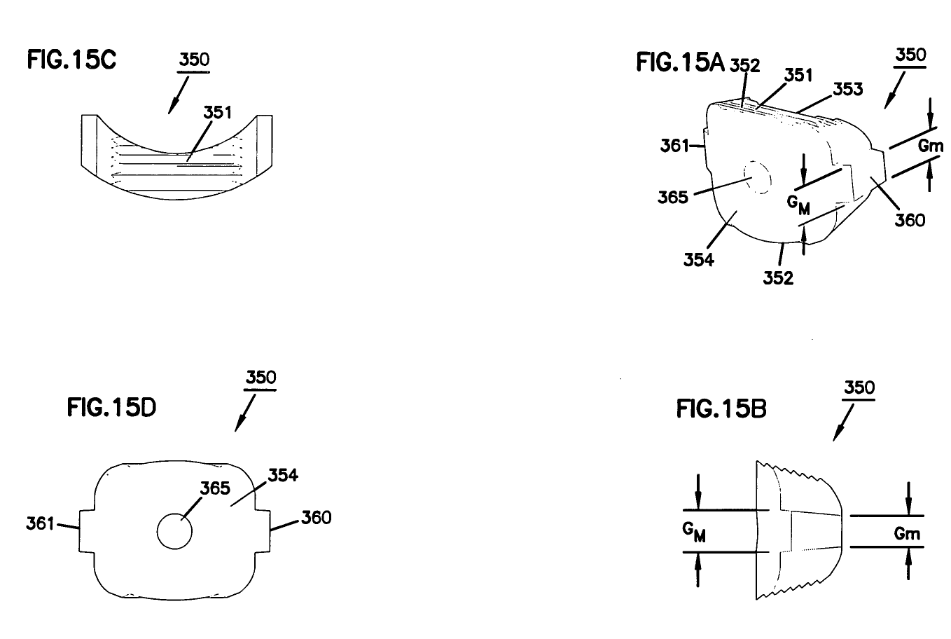

FIGS. 14a-14f and 15a-15d illustrate an alternative embodiment of an implant arrangement according to the invention. In the illustrated embodiment, implant 320 is shown with a growth component 321, such as cancellous bone 322.

Implant 320 includes an outer wall 323, an inner wall 324 and has a generally “C-shaped” configuration with a first arm 325 continuous with a second arm 326 and a void 327 therebetween. In addition, implant 320 includes a first bearing surface 328 and a second bearing surface 329. In the illustrated embodiment, the first bearing surface 328 includes an engaging surface 330, comprising ridges 330a, and a second bearing surface 329 having an engaging surface 331 comprising ridges 331a. As discussed previously, an engaging surface reduces the likelihood of post-implantation mobility of an implant.

As illustrated best in FIG. 14c, in this embodiment, implant 320 has a major height H_{M} and minor height Hₘ. This tapered configuration could also be provided in other implants discussed herein, such as implants 120 and 140.

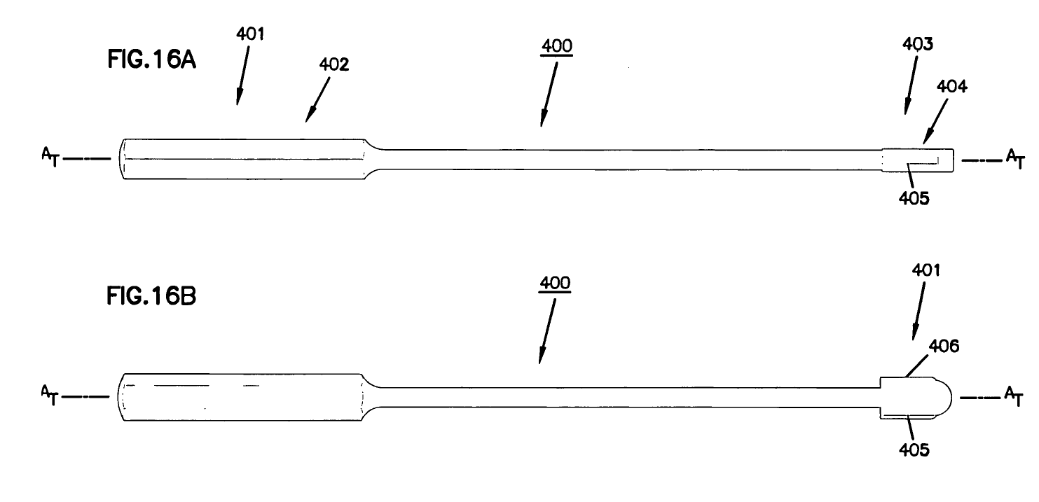

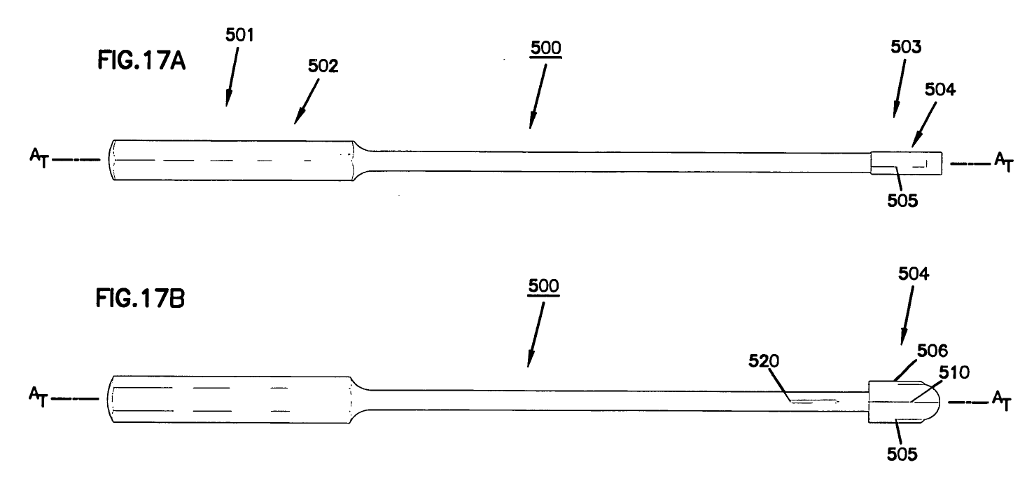

FIGS. 14e and 14f illustrate implant 320 without growth component 321. As can be seen, inner wall 324 includes a first groove 336 extending partially along first arm 325 and a second groove 337 extending partially along second arm 326. Although grooves 336 and 337 are shown as being discontinuous, the groove can be continuous around inner wall 324. As will be described below, grooves 336 and 337 provide for attachment of a cover 350 (FIGS. 15a-15d) or an implant insertion tool 400, 500 (FIGS. 16a, 16b, 17a, 17b).

FIGS. 15a-15d illustrate a cap for positioning in void 327 between arms 325 and 326. In the illustrated embodiment, cap 350 has a first bearing surface 351, a second bearing surface 352, an inner surface 353 and an outer surface 354. Bearing surface 351 includes an engaging surface 352 which can be similar to that of implant 320 (bearing surface 352 can also include an engaging surface). On each side, cap 350 also includes a tab 360 and 361. Tabs 360 and 361 are configured to pass into grooves 337 and 336. As illustrated in FIGS. 15a and 15b, tab 360 (and 361) have a major height G_{M}, and minor height Gₘ. The difference in height G_{M} and Gₘ provides tabs 360 and 361 with a diverging taper from inner surface 353 to outer surface 354. Thus, when tabs 360 and 361 have passed into grooves 337 and 336 as cap 350 is advanced within arms 325, 326 the taper from height Gₘ to height Gₘ is selected to provide for a snug fit between tabs 360 and 361 and grooves 336 and 337 to retain cap 350 in position. That is, cap 350 is friction fit into implant 320. The grooves 336 and 337 of implant 320, and a cap, such as cap 350 can be used with other implants, such as implants 120 and 140.

Cap 350 can also include a bore 365 that may be threaded (not shown) which permits for attachment of an insertion tool having a threaded plate end to mate with bore 365.

FIGS. 16a and 16b illustrate one embodiment of an implant insertion tool 400 suitable for use with an implant of the invention. As illustrated, implant insertion tool 400 has a proximal end 401 including a handle 402 for operating the instrument and a distal end 403 having a working end 404. Working end 404 include tabs 405 and 406 that fit cooperatively within grooves 336 and 337 of implant 320. Thus, implant 320 can be mounted at the working end 404 of implant insertion tool 400 allowing the surgeon to manipulate implant 320 via tool 400 into a suitable position at the fusion site.

FIGS. 17a and 17b illustrate an alternative embodiment of an implant insertion tool 500. As described above, for implant insertion tool 400, implant insertion tool 500 includes a proximal end 501 having a handle 502 and a distal end 503 including a working end 504. Insertion tool 500 includes tabs 506 and 505 which cooperatively fit within grooves 336 and 337 of implant 320. In addition, the working end 504 of implant insertion tool 500 includes a slot 510 which permits tabs 506 and 505 to expand laterally away from axis A_{T}. In a typical embodiment, expansion of tabs 506 and 505 away from axis A_{T} is the normal position. A sleeve (not shown) can then be slid from the proximal end 520 of slot 510 to force tabs 505 and 506 towards axis A_{T}. That is, when the sleeve is advanced distally it brings tabs 505 and 506 together towards axis A_{T}. In this position, the working end 504 of implant insertion tool 500 can be inserted into grooves 336 and 337. The sleeve can then be slid towards the proximal end 520 to allow tabs 505 and 506 to expand laterally away from axis A_{T} to provide friction holding of implant 320 on working end 504. After placement of implant 320, the sleeve can be slid distally to bring tabs 505 and 506 back together at axis A_{T} to remove implant insertion tool 500. Other arrangements providing for expansion and contraction of tabs 505, 506, relative to axis A_{T} will be appreciated after reading this disclosure.

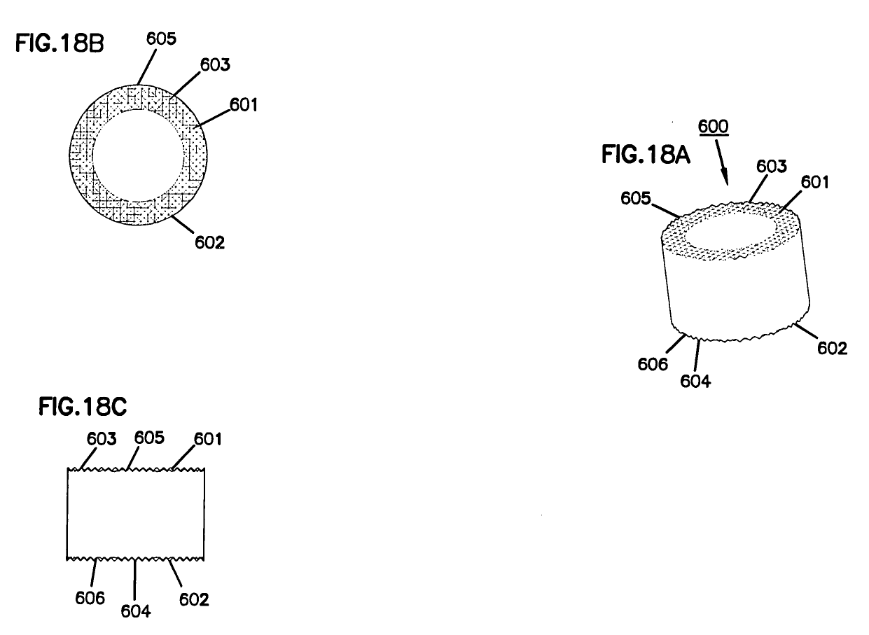

FIGS. 18a-18c illustrate an implant 600 having a first bearing surface 601 and a second bearing surface 602. In the illustrated embodiment, bearing surface 601 (and 602) include engaging surfaces 603 and 604. Bach of engaging surfaces 603 and 604 include knurls 605 and 606. This is one alternative embodiment of an engaging surface that can be used on any of the implants disclosed herein.

The implants described herein can be included in a kit comprising a plurality of incrementally sized implants which can be selected for use by the clinician based on the size needed for a particular patient. In other embodiments kits will be provided which include instrumentation for performing an implant procedure with or without a plurality of incrementally sized implants.

Having now described the present invention, it will be apparent to one of ordinary skill in the art that many changes and modifications can be made in the invention without departing from the spirit or scope of the appended claims.

We claim:

1. A bone implant for fusion of bone, said implant comprising:
a first support portion having a first exterior side;
a second support portion having a second exterior side, the second support portion being located opposite the first support portion;
a growth portion located between the first support portion and the second support portion;
a first end and a second end, the second end being located opposite the first end;
a first bearing surface and a second bearing surface, the second bearing surface being located opposite the first bearing surface; the first bearing surface and the second bearing surface having a length that extends from the first end to the second end;
the first end and the second end having a height that extends from the first exterior side of the first support portion to the second exterior side of the second support portion, the height being less than the length of the first bearing surface and the second bearing surface to facilitate insertion of the implant between two bones in a height-wise orientation such that the exterior sides of the first and second support portions are adjacent endplates of the two bones; and
wherein upon rotation of the implant, the implant is oriented length-wise such that the first bearing surface and the second bearing surface are adjacent the endplates of the two bones.

2. The bone implant according to claim 1 wherein the support portion is cortical bone and the growth portion is cancerous bone.

3. The bone implant according to claim 2 wherein the implant is formed of cranium bone.

4. The bone implant according to claim 1 wherein the first bearing surface is convex.

5. The bone implant according to claim 1 wherein the first and second exterior sides of corresponding first and second support portions are substantially planar.

6. A bone implant for fusion of bone, said implant comprising:
a first bearing surface including a first portion of cortical cancellous bone;
a second bearing surface including a second portion of cortical cancellous bone, the second bearing surface being located opposite the first bearing surface; the first bearing surface and the second bearing surface having a length;
a first cortical surface and a second cortical surface opposite the first cortical surface;
a first end and a second end opposite the first end, the first end and the second end having a height that extends from the first cortical surface to the second cortical surface;
wherein the height of the first and second ends is less than the length of the first and second bearing surfaces to accommodate insertion of the implant at a gap between two bones such that the first and second cortical surfaces contact opposing endplates of the two bones; and
wherein upon rotation of the implant, the implant is oriented in a length-wise position to expand the gap and position the implant such that first and second bearing surfaces contact the opposing endplates of the two bones.

7. A bone implant for fusion of bone, said implant comprising:
an implant body including a first load bearing end positioned opposite from a second load bearing end, the implant body defining a length that extends between the first and second load bearing ends;
the implant body including first and second support components that extend from the first load bearing end to the second load bearing end;
the implant body including a growth component positioned between the first and second support components, the growth component extending from the first load bearing end to the second load bearing end;
the implant body defining a height that extends between the first and second support components, the height being less than the length to accommodate insertion of the implant body between endplates of two bones in a height-wise orientation; and
wherein upon rotation of the implant body, the implant body is oriented length-wise such that the first load bearing end and the second load bearing end are adjacent the endplates of the two bones.

8. A bone implant for fusion of bone, said implant comprising:
a support component; and
a growth component.

9. The bone implant according to claim 8 wherein the support component is adjacent the growth component.

10. The bone implant according to claim 8 wherein the support component comprises cortical bone.

11. The bone implant according to claim 8 wherein the growth component comprises cancellous bone.

12. The bone implant according to claim 8 wherein the growth component is not surrounded entirely by the support component.

13. The bone implant according to claim 8 wherein the growth component has a rectilinear configuration and the support component has a rectilinear configuration.

14. The bone implant according to claim 8 wherein the support component has a circular configuration.

15. The bone implant according to claim 8 further comprising a pin and wherein:
the growth component includes a first bore,
the support component includes a second bore, and
alignment of the first bore and the second bore forms a channel for passing the pin therethrough to maintain the growth component and support component in a fixed relationship.

16. The bone implant according to claim 10 wherein the support component has a “C” shape comprising a first arm continuous with a second arm and having a gap therebetween and the growth component is receivable within the gap.

17. The bone implant according to claim 8 wherein the support component has at least one bearing surface including an engaging surface.

18. The bone implant according to claim 8 wherein the support component of the implant is a synthetic material.

19. The bone implant according to claim 18 wherein the support component of the implant is manufactured from porous titanium.

20. The bone implant according to claim 8 wherein the implant comprises cranial bone having a cortical support component and a cancellous growth component.

21. A bone implant for fusion of bone, the implant comprising:
a support component having a first bearing surface and a second bearing surface, and further including at least one bore passing through the implant.

22. A bone implant for fusion of bone, the implant comprising:
a circular support component.

23. A kit for fusing an intervertebral disc space between a first and second vertebrae, kit comprising:
an implant body having a support component; and
an implant insertion tool.

24. A method for fusing an intervertebral disc space between a first and second vertebrae, the method comprising a step of:
identifying a first and second vertebrae to be fused;
selecting a bone implant harvested from cranial bone; and
inserting the bone implant between the first and second vertebrae.

25. The method according to claim 24 wherein the cranial bone includes a cortical portion and a cancerous portion.

26. The method according to claim 24 wherein the implant is configured to follow contours of an exterior surface of the first and second vertebrae.

27. The method according to claim 24 wherein the cranial bone includes a cancellous region and a first cortical region adjacent a first side of the cancellous region and a second cortical region adjacent a second side of the cancellous region.
